(19) United States
(12) Patent Application Publication
(10) Pub. No.: US 2012/0109303 A1
(43) Pub. Date: May 3, 2012

(54) IMPLANT ASSEMBLIES, DEVICES AND METHODS FOR PROVIDING STABILIZATION BETWEEN FIRST AND SECOND VERTEBRAE

(75) Inventor: Cristian Capote, Memphis, TN (US)

(73) Assignee: Warsaw Orthopedic, Inc., Warsaw, IN (US)

(21) Appl. No.: 12/914,702

(22) Filed: Oct. 28, 2010

(51) Int. Cl. A61F 2/44

(52) U.S. Cl. 623/17.12

(57) ABSTRACT

Systems, methods and devices for providing stabilization between first and second vertebrae are provided. More particularly, in one form an implant assembly includes first and second end members configured to engage with the first and second vertebrae, respectively, and a support body configured to extend between and engage with the end members. In one aspect of this form, one or both of the end members is configured to facilitate injection of a material into one or both of the vertebrae. In another aspect, one or both of the end members includes a mesh material extending across a hollow interior to facilitate bone growth through the end member toward the support body. In another form, techniques for providing stabilization and support to vertebrae on opposite sides of a space created by removal of one or more vertebral elements are provided. However, different forms and applications are also envisioned.

### BACKGROUND

The present application relates to implant assemblies, systems, methods and devices for providing stabilization and support between first and second vertebrae, and more particularly but not exclusively relates to implant assemblies, systems, devices and techniques for replacing one or more anatomical features located between the first and second vertebrae.

A variety of spinal injuries and deformities can occur due to trauma, disease, or congenital effects. These injuries and diseases can, ultimately, result in the complete or partial destruction of one or more vertebral bodies and lead to a vertebrectomy in which the one or more damaged vertebral bodies and their adjacent discs are excised or a corpectomy in which an anterior portion of the one or more damaged vertebral bodies and their adjacent discs are excised. Reconstruction of the spine following the vertebrectomy or corpectomy can present a number of challenges for the surgeon.

One surgical concern is securely interposing an intervertebral implant between the remaining vertebral bodies to ensure that the position of the implant is maintained following completion of the surgical procedure. One technique for promoting secure positioning of the intervertebral implant between the vertebrae involves the use of bone grafts or other bone-growth promoting materials to influence fusion between the vertebrae. However, positioning and maintaining these bone grafts or other bone-growth promoting materials in contact with or close proximity to the vertebrae in a manner likely to increase the likelihood of fusion can be difficult. In addition, in some cases fusion across the space between the remaining vertebrae may not be prescribed by the surgeon or physician.

Moreover, in some instances the ability to securely maintain the positioning of the intervertebral implant becomes more complex when the integrity of the bone of one or both of the adjacent vertebrae has been compromised due to disease or degeneration. For example, the diseased or degenerated state of the bone can lead to faulty purchase of a bone engaging member used to anchor the intervertebral implant and/or subsidence of the intervertebral implant into one or both of the adjacent vertebrae. In addition, if not addressed the diseased or degenerated state of the one or more adjacent vertebrae can undesirably lead to subsequent surgical procedures in which the vertebra(e) is(are) removed and replaced, amongst other possibilities.

Thus, there remains a need for further improvements in the implants, devices, instruments, assemblies, apparatuses, systems, and methods for providing stabilization and support to the remaining vertebrae on opposite sides of the space created during a procedure in which one or more vertebral elements positioned between the vertebrae are removed.

### SUMMARY

Implant assemblies, systems, methods and devices for providing stabilization between first and second vertebrae are provided. More particularly, in one form an implant configured to be positioned in a space created by the removal of one or more vertebral elements and to engage the vertebrae on opposite sides of the space is provided. In one form, in addition to filling the space between the vertebrae, the implant includes one or more bone anchor elements configured to engage with the vertebrae and through which a material can be injected into the vertebrae to reinforce the vertebrae and/or to enhance engagement of the bone anchor elements with the vertebrae. In addition, in this or another form the implant can also include one or more features configured to facilitate ingrowth of bone or bony tissue into or around the implant, and/or to promote bone growth across the space to fuse the vertebrae on the opposite sides of the space together. In one aspect, the implant includes an elastic body that facilitates dynamic stabilization and support of the vertebrae, although forms where the implant includes a rigid body are also included. However, different forms and applications are also envisioned.

In one embodiment, an implant includes first and second end members configured to engage with first and second vertebrae, respectively, positioned on opposite sides of a space formed by removing one or more vertebral elements. The implant also includes a support body configured to extend between and engage with the end members. In one form of this embodiment, one or both of the end members is configured to facilitate injection of a material, such as bone cement, into one or both of the vertebrae. In another form, one or both of the end members includes a mesh material extending across a hollow interior to support a bone graft or bone growth promoting material, and also facilitate bone growth through the end member toward the support body. In another form, techniques for providing stabilization and support to vertebrae on opposite sides of a space created by removal of one or more vertebral elements are provided.

In another embodiment, an implant assembly configured to be positioned between a first vertebra and a second vertebra includes a first end member including at least one bone engaging portion configured to engage with the first vertebra; a second end member including at least one bone engaging portion configured to engage with the second vertebra; and a support body configured to axially extend between and engage with the first and second end members. In one form of this embodiment, at least the first end member includes a first injection port in fluid communication with the at least one bone engaging portion, and the at least one bone engaging portion of the first end member includes a number of fenestrations through which a material injected into the first injection port is deliverable into the first vertebra. In a further aspect of this form, the second end member also includes a second injection port in fluid communication with the at least one bone engaging portion, and the at least one bone engaging portion of the second end member includes a number of fenestrations through which a material injected into the second injection port is deliverable into the second vertebra.

In yet another embodiment, an implant assembly configured to be positioned between a first vertebra and a second vertebra includes a first end member including at least one bone engaging member configured to engage with the first vertebra; a second end member including at least one bone engaging member configured to engage with the second vertebra; and a support body configured to axially extend between and engage with the first and second end members. In one form, at least the first end member includes a base portion extending between a first side facing the second end member and a second side from which the at least one bone engaging member extends. The base portion further includes a hollow interior extending between and opening at the first and second sides and a mesh material extending across the hollow interior. In a further aspect of this form, the second end member also includes a base portion extending between a third side facing the first end member and a fourth side from which the at least one bone engaging member extends. The base portion of the second end member also includes a hollow interior extending between and opening at the third and fourth sides and a mesh material extending across the hollow interior.

In still another embodiment, a method for positioning an implant between a first vertebra and a second vertebra includes providing a first end member including at least one bone engaging portion configured to engage with the first vertebra, a second end member including at least one bone engaging portion configured to engage with the second vertebra, and a support body configured to axially extend between and engage with the first and second end members. The method also includes positioning the first end member into a space between the first vertebra and the second vertebra and engaging the first end member with the first vertebra; positioning the second end member into the space between the first vertebra and the second vertebra and engaging the second end member with the second vertebra; injecting a material into the first end member and delivering the material into the first vertebra through the at least one bone engaging portion of the first end member; and positioning the support body between and into engagement with the first and second end members. In one aspect of this method, injecting the material into the first end member occurs before positioning the support body, although aspects where injecting the material into the first end member occurs after positioning the support body are also contemplated.

In another embodiment, techniques for replacing one or more removed vertebral elements and reinforcing one or both of the remaining vertebrae on opposite sides of the removed vertebral elements include positioning an implant between the vertebrae, engaging a portion of the implant in each vertebra, and injecting a vertebral body treatment material or bone filling material into one or both of the vertebrae through the portion of the implant engaged therein.

Another embodiment of the present application comprises a unique implant assembly for providing stabilization and/or support between vertebrae positioned on opposite sides of a space created by a vertebrectomy or corpectomy, although use of the implant in a space created by a discectomy are also contemplated. An additional embodiment of the present application comprises a unique system that utilizes a single implant for providing stabilization and support to a pair of vertebral bodies and for reinforcing one or more of the vertebral bodies with an injectable material. In one aspect of these and the foregoing embodiments, the implant or implant assembly can be positioned between the vertebrae in a compacted or unexpanded configuration and then expanded to engage with the vertebrae.

Other embodiments include unique methods, systems, devices, kits, assemblies, equipment, and/or apparatus for use in connection with the stabilization and support, or the stabilization, support and reinforcement, of a pair of vertebrae. However, in other embodiments, different forms and applications are also envisioned.

Further embodiments, forms, features, aspects, benefits, objects and advantages of the present application will become apparent from the detailed description and figures provided herewith.

### BRIEF DESCRIPTION OF THE FIGURES

FIG. 1 is a side view of a segment of the spinal column.

FIG. 2 is an enlarged, side view of a portion of the spinal column segment illustrated in FIG. 1 with an intervertebral implant assembly positioned between a pair of vertebrae.

FIG. 3 is a perspective view of an end member of the implant assembly illustrated in FIG. 2.

FIG. 4 is an end view of the end member illustrated in FIG. 3.

FIG. 5 is a side plan view of a support body of the implant assembly illustrated in FIG. 2 in a deflated configuration.

FIG. 6 is a perspective view of an insertion device used for positioning the end members of the assembly illustrated in FIG. 2 into engagement with the vertebrae.

FIG. 7 is a side plan view of one technique for injecting a material into the vertebrae following engagement of the end members therewith.

FIG. 8 is a side plan view of one technique for inflating the support body of the implant assembly of FIG. 2.

FIG. 9 is a side plan view of an alternative embodiment support body engaged with the end members and positioned between the vertebrae.

FIG. 10 is a side plan view of another alternative embodiment support body engaged with the end members and positioned between the vertebrae.

FIG. 11 is a perspective view of another alternative embodiment support body configured to engage with the end members of the implant assembly illustrated in FIG. 2.

FIG. 12 is a side plan view of an alternative embodiment end member.

FIG. 13 is a top plan view of the end member illustrated in FIG. 12.

FIG. 14 is a section view of the end member illustrated in FIG. 12 taken along view line 13-13.

FIG. 15 is a side plan view of an implant assembly including end members illustrated in FIGS. 12-14 engaged between a pair of vertebrae.

FIG. 16 is a side plan view of another alternative embodiment end member.

FIG. 17 is a top plan view of another alternative embodiment end member.

FIG. 18 is a section of view of the end member illustrated in FIG. 17 taken along view line 18-18.

FIG. 19 is a section view of another alternative embodiment end member.

FIG. 20 is a top plan view of another alternative embodiment end member.

FIG. 21 is a section view of the end member illustrated in FIG. 20 taken along view line 20-20.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

Instruments, devices, systems, apparatuses, assemblies, techniques and methods for performing spinal surgery, including but not limited to stabilization and support, or stabilization, support and reinforcement, of a pair of vertebrae are provided. More particularly, in one form an implant configured to be positioned in a space created by the removal of one or more vertebral elements and to engage the vertebrae on opposite sides of the space is provided. In one aspect of this form, the implant includes a length sized to extend between and engage with an endplate of each of the vertebrae. In addition, the implant also includes one or more bone anchor elements configured to engage with the vertebrae and, in one form, facilitate injection of a filler material into one or both of the vertebrae to provide reinforcement to the vertebrae and/or to enhance the engagement of the bone anchor elements with the vertebrae. In addition, in this or another form the implant can also include one or more features configured to facilitate ingrowth of bone or bony tissue into or around the implant, and/or to promote bone growth across the space to fuse the vertebrae on the opposites sides of the space together. In one aspect, the implant includes an elastic body that facilitates dynamic stabilization and support of the vertebrae, although forms where the implant includes a rigid body are also included. In another form, an implant is configured to be positioned between a pair of vertebrae and replace one or more diseased or degenerated vertebral elements that have been removed from between the vertebrae, and further facilitate injection of a filler material from an intervertebral location into one or both of the vertebrae to provide reinforcement to the bone or bony tissue of the remaining vertebrae. Still, it should be appreciated that alternative forms, aspects, configurations, arrangements and methods are contemplated with respect to the subject matter disclosed and described herein.

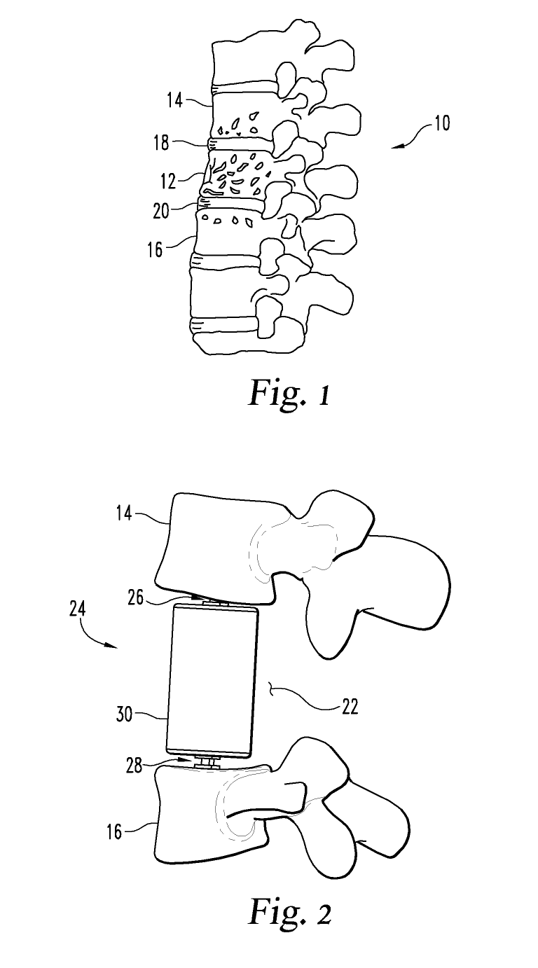

Referring now generally to FIG. 1, there is illustrated a segment of the spinal column 10 with a damaged vertebra 12 positioned between vertebrae 14 and 16, which are also partially damaged. An intervertebral disc 18 extends between vertebrae 12 and 14, and an intervertebral disc 20 extends between vertebrae 12 and 16. In the illustrated form, vertebra 12 is damaged to such an extent that its removal and replacement are necessary. However, the damage to vertebrae 14, 16 does not warrant their removal and is of a nature that can be addressed by reinforcement of the vertebrae with a filler material. In a typical surgical procedure for removing vertebra 12, it is removed together with discs 18, 20 thereby creating a void or space 22 between vertebrae 14, 16 as illustrated in FIG. 2. This procedure may be performed using an anterior, anterolateral, or other approach known to those skilled in the art. A vertebral implant assembly as described in greater detail below is then positioned between and engaged with the vertebrae 14, 16. Although use of the implant assemblies disclosed herein are described in connection with removal of a single vertebra and the adjacent discs, it should be appreciated that the disclosed implant assemblies could also be used in connection with the removal of two or more vertebrae and the corresponding discs. It is also contemplated that the disclosed implant assemblies could be inserted into an intervertebral disc space without the removal of any vertebrae. In addition, while use of the disclosed implant assemblies is described in connection with vertebrae 14, 16 which are also partially damaged, it should be appreciated that use of the implant assemblies in a space where only one or neither of vertebrae 14, 16 is damaged is also contemplated.

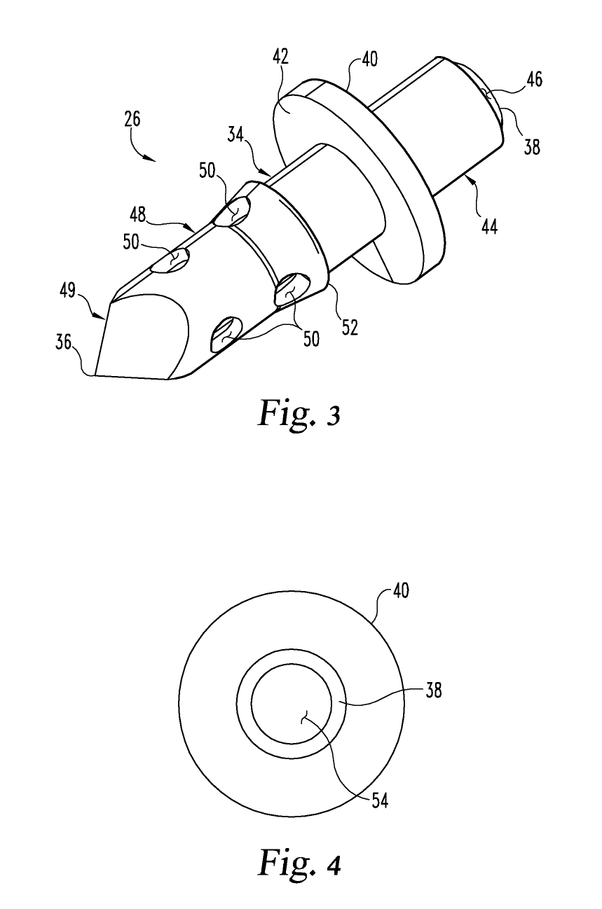

Following removal of vertebra 12, an implant assembly 24 is positioned in space 22 as illustrated in FIG. 2 for example. Implant assembly 24 generally includes a first end member 26, a second end member 28, and a support body 30 configured to extend between and engage with first and second end members 26, 28. Further details regarding first and second end members 26, 28 and support body 30 will be provided below in connection with FIGS. 3-5. More particularly, an enlarged, perspective view and an enlarged, end view of end member 26 are illustrated in FIGS. 3 and 4, respectively. End member 26 includes an elongated body 34 that extends between a first end 36 and a second end 38. A radial flange 40 is positioned between first end 36 and second end 38 and generally separates a coupling portion 44 and a bone engaging portion 48. Radial flange 40 includes an end surface 42 that faces toward bone engaging portion 48 and is configured to bear against the endplate of vertebra 14 when bone engaging portion 48 is engaged with vertebra 14. While not illustrated, it should be appreciated that end surface 42 of flange 40 can be provided with one or more bone engaging features such as teeth or spikes, just to provide a few non-limiting examples. Coupling portion 44 is generally configured to engage with a coupling portion of support body 30, further details of which will be provided below. Similarly, second end 38 of elongated body 34 includes a chamfer 46 configured to guide coupling portion 44 into engagement with support body 30.

Bone engaging portion 48 includes a pointed tip 49 that is positioned adjacent to first end 36 and configured to facilitate insertion of bone engaging portion 48 into vertebra 14. Bone engaging portion 48 also includes a radially extending barbed portion 52 configured to resist expulsion of end member 26 from vertebra 14 following its engagement therewith. In addition to or in lieu of barbed portion 52, it is also contemplated that bone engaging portion 48 could be provided with other features to resist expulsion from and/or engage with vertebra 14, non-limiting examples of which include threads, ridges, grooves, knurling or other surface roughening, just to provide a few possibilities. Bone engaging portion 48 also includes a plurality of fenestrations 50 that are in fluid communication with a hollow interior 54 that opens through second end 38 of elongated body 34. Similarly, hollow interior 54 serves as an injection port at second end 38 such that an injectable material can be injected into hollow interior 54 at second end 38 and passed through fenestrations 50 into vertebra 14, further details of which will be provided below. In other non-illustrated forms, end member 26 could include an injection port that opens through coupling portion 44 or bone engaging portion 48 rather than through second end 38, just to provide a few non-limiting possibilities.

While not previously discussed, it should be appreciated that second end member 28 will generally include the same or a substantially similar configuration as that described for first end member 26 above, although forms where end members do not include the same or substantially similar configurations are also contemplated. For example, in one non-illustrated form, only one of end members 26, 28 is provided with a hollow interior and fenestrations through which an injectable material is delivered to the corresponding vertebrae 14, 16.

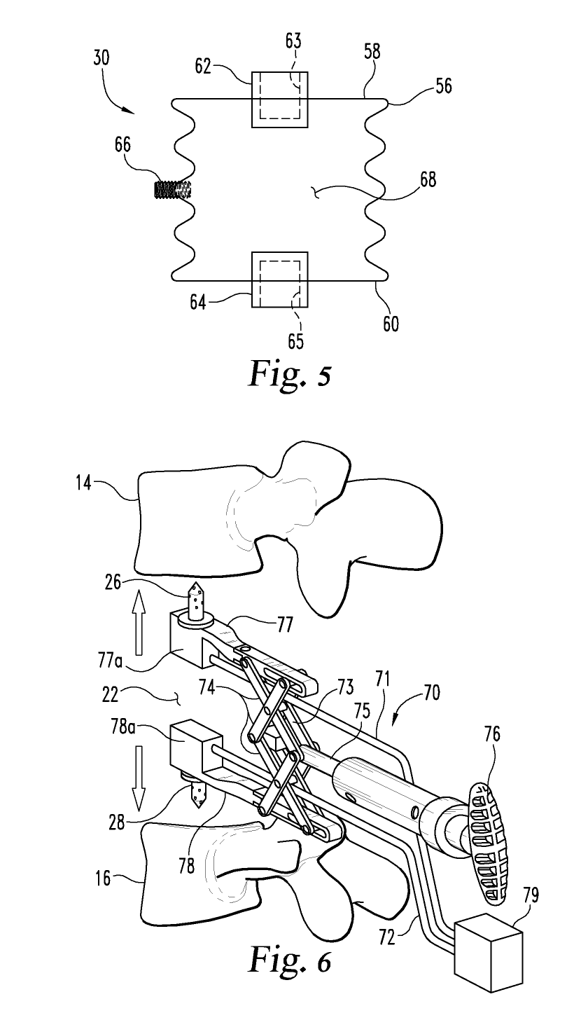

Turning now to FIG. 5, support body 30 is illustrated in a side plan view in a deflated or unexpanded configuration. More particularly, support body 30 is formed of a balloon-like structure 56, such as a flexible container or bag, that includes a hollow interior 68 into which a material may be injected in order to expand support body 30 to the inflated or expanded configuration illustrated in FIG. 2 for example. Balloon-like structure 56 extends between a first end 58 and a second end 60 that are positioned adjacent to first and second end members 26, 28, respectively, when balloon-like structure 56 is in the expanded configuration. First end 58 includes a coupling member 62 configured to engage with first end member 26 and second end 60 includes a coupling member 64 configured to engage with second end member 28. More particularly, coupling members 62, 64 each include an internal receptacle 63, 65, respectively, within which the coupling portions of first and second end members 26, 28 can be positioned. In one form, first and second end members 26, 28 are engaged with coupling members 62, 64 via a friction or press fit arrangement, although other forms for facilitating engagement between first and second end members and coupling members 62, 64 are contemplated. For example, in one non-limiting form, first and second end members 26, 28 can include external threading configured to engage with internal threading of coupling members 62, 64. In another non-limiting form, one or both of coupling members 62, 64 can include a transverse set screw configured to bear against first and second end members 26, 28 in internal receptacles 63, 65 in order to prevent disengagement of coupling members 62, 64 from first and second end members 26, 28.

Balloon-like structure 56 may be constructed of a complaint biocompatible material, such as a resin or polymer that may include materials such as nylon, polyethylene, polyurethane, silicone, polyethylene, polypropylene, polyimide, polyamide, and polyetheretherketone (PEEK). Balloon-like structure 56 may be formed from materials that are used in other conventionally known biomedical applications, such as balloon angioplasty. Further, balloon-like structure 56 may be reinforced with concentric layers of similar or dissimilar materials and/or fabrics (not specifically shown). For instance, a reinforcing structure may be constructed of a wide variety of woven or nonwoven fibers, fabrics, metal mesh such as woven or braided wires, polymeric fibers, ceramic fibers, and carbon fibers. Biocompatible fabrics or sheet material such as ePTFE and Dacron®, Spectra®, and Kevlar® may also be used. In addition, balloon-like structure 56 may be formed from a solid material or an air-permeable material. For instance, when formed from an air-permeable material, balloon-like structure 56 can be formed of a mesh material that facilitates bony ingrowth into support body 30 and/or bone formation throughout support body 30 in order to provide a bony fusion between vertebrae 14, 16.

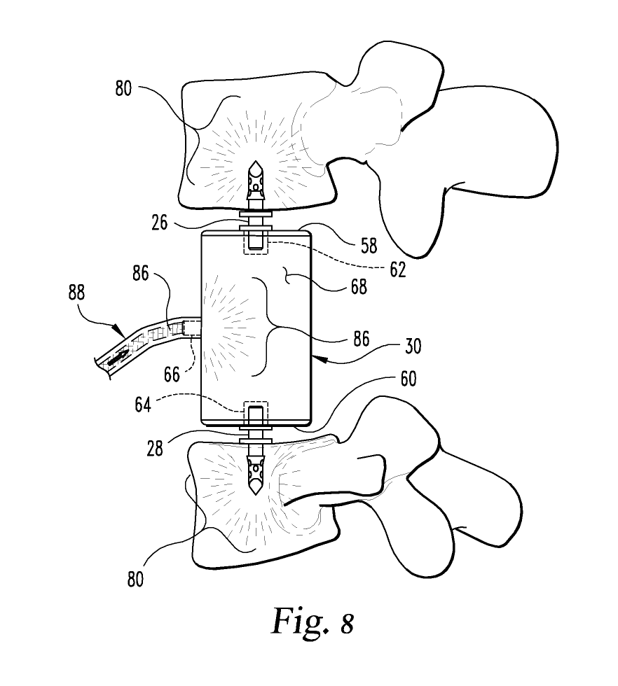

Various techniques may be used to introduce an injectable substance or material into balloon-like structure 56. In the embodiment shown, a injection port 66 is provided on balloon-like structure 56. Notably, while only one injection port 66 is depicted, additional ports may be used. Further, port 66 may be located in different locations depending on a particular implementation and angle of approach. Injection port 66 may be attached to a syringe or other pumping mechanism (see FIG. 8) to fill balloon-like structure 56. An injectable substance may flow through the injection port 66 into hollow interior 68 of balloon-like structure 56. As the injectable substance fills balloon-like structure 56, ends 58, 60 of balloon-like structure 56 move away from one another in the direction of end members 26, 28, respectively. As hollow interior 68 of balloon-like structure 56 is filled and support body 30 expands, a displacement force can be applied to end members 26, 28 that cause them to separate from one another. In one form, injection port 66 may include a self-sealing valve (not specifically shown) that prevents the injectable substance or material from flowing in one direction or another once the balloon-like structure 56 is filled.

A variety of injectable substances 86 (FIG. 8) may be inserted into balloon-like structure 56 in order to expand support body 30. In one form, injectable substance 86 is at least partially compliant or elastic following injection into support body 30 such that dynamic stabilization is provided between vertebrae 14, 16. However, in other forms, injectable substance 86 can harden into a rigid state following its injection into support body 30 such that rigid stabilization is provided between vertebrae 14, 16. In one embodiment, injectable substance 86 is a fluid, such as a gas or a liquid. In another embodiment, injectable substance 86 can be a solid, such as a powder. In one form, injectable substance 86 inserted into support body 30 is a bone cement, bone filling material, bone treatment material, or bone-growth promoting material. In one embodiment, injectable substance 86 is a curable liquid that solidifies after a predetermined amount of time or under the influence of an external catalyst. For instance, an injectable liquid may cure under the influence of heat or light, including ultraviolet light. Some non-limiting examples of in situ curable liquids include epoxy, PMMA, polyurethane, and silicone. A curable substance may cure to a substantially rigid state or to a flexible, but relatively incompressible state.

Referring now to FIGS. 6-8, further details regarding the positioning of implant assembly 24 between vertebrae 14, 16 will be provided. More particularly, in FIG. 6 end members 26, 28 are engaged with an insertion device 70 constructed to position end members 26, 28 relative to vertebrae 14, 16. Device 70 includes first and second arms 77, 78 that include engaging portions 77a, 78a, respectively, sized to hold one of end members 26, 28. An adjustment mechanism 73 controls the distance between arms 77, 78. In this embodiment, adjustment mechanism 73 is a jack device having pivoting linkages 74 attached to an arm 75. Handle 76 is operatively connected to arm 75 to control the movement of linkages 74 and thus the relative spacing of arms 77, 78.

In use, end members 26, 28 are engaged with arms 77, 78 with their bone engaging portions extending toward vertebrae 14, 16 in preparation for their engagement therewith. With end members 26, 28 engaged with arms 77, 78, arms 77, 78 are positioned in a relatively closed orientation and are spaced apart a distance to fit between vertebrae 14, 16. The surgeon then manipulates handle 76 to insert arms 77, 78 with end members 26, 28 between vertebrae 14, 16. Once inserted, handle 76 is rotated to move apart arms 77, 78. This movement causes the bone engaging portions of end members 26, 28 to be driven into vertebrae 14, 16 to attach end members 26, 28 therewith. The expansion movement may also distract vertebrae 14, 16 as desired.

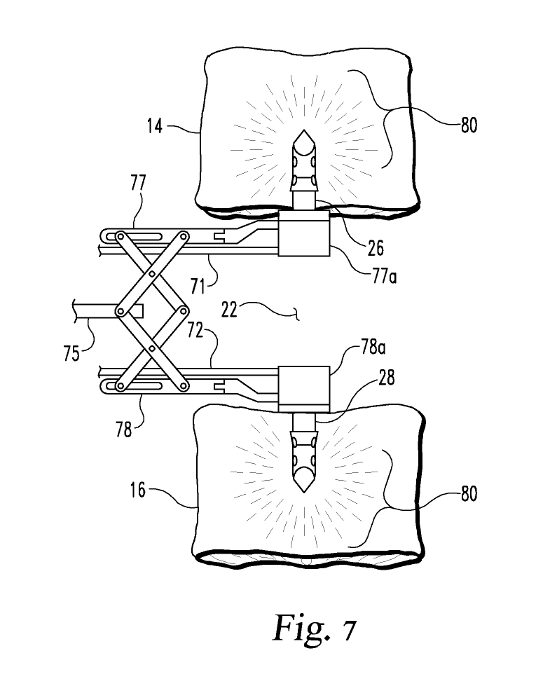

Device 70 also includes a pair of conduits 71, 72 that extend between and engage with engaging portions 77a, 78a and an injectable material source 79. Engaging portions 77a, 78a are configured to direct an injectable material provided from source 79 into and through end members 26, 28 into one or both of vertebrae 14, 16. Similarly, following engagement of end members 26, 28 with vertebrae 14, 16, an injectable material 80 from injectable material source 79 can be passed through conduits 71, 72 into engaging portions 77a, 78a where injectable material 80 is injected through end members 26, 28 into vertebrae 14, 16. While not being limited to any particular form, injectable material 80 may be injected into one or both of vertebrae 14, 16 in order to strengthen or treat diseased or damage bone of vertebrae 14, 16 due to compressive trauma, fractures or osteoporosis (among other possibilities), enhance engagement of end members 26, 28 with vertebrae 14, 16, reinforce bone adjacent to prosthetic implants, and/or repair bone loss caused by implantation or revision of prosthetic systems. More particularly, as illustrated in FIG. 7, injectable material 80 has been injected into vertebrae 14, 16 through the fenestrations of end members 26, 28. In other non-illustrated forms, it is contemplated that only one of end members 26, 28 is injected with injectable material 80.

While not previously mentioned, it should be appreciated that engaging portions 77a, 78a can be engaged with the coupling portions of end members 26, 28 in a sealed configuration in order to prevent leakage of injectable material 80 into space 22 and/or to allow injectable material 80 to be injected under pressure so that it can be sufficiently dispersed throughout vertebrae 14, 16 as appropriate in light of the condition of vertebrae 14, 16 being treated with injectable material 80. In addition, while not illustrated it should be appreciated that one or both of end members 26, 28 may include a self-sealing valve that prevents injectable material 80 from flowing back into space 22 once it has been injected into vertebrae 14, 16. Further, while not intending to be limited to any particular form, it is contemplated that insertion device 70 could include a pump or syringe-type mechanism configured to deliver injectable material 80 from source 79 to engaging portions 77a, 78a, although other possibilities for delivering injectable material 80 exist. Once end members 26, 28 are engaged with vertebrae 14, 16 and injectable material 80 has been injected into vertebrae 14, 16 as desired, insertion device 70 can be disengaged from end members 26, 28 and removed from the surgical site.

In other non-illustrated forms, it is contemplated that conduits 71, 72 may be absent from insertion device 70 and engaging portions 77a, 78a may otherwise not be configured to inject injectable material 80 into end members 26, 28. Similarly, in these forms a separate injection device can be engaged with one or both of end members 26, 28 following their engagement with vertebrae 14, 16 and used to inject injectable material 80 into one or both of vertebrae 14, 16. As discussed above, it should be appreciated that the injection device can be engaged with the coupling portions of end members 26, 28 in a sealed configuration in order to prevent leakage of injectable material 80 into space 22 and/or to allow injection material 80 to be injected under pressure so that it can be sufficiently dispersed throughout vertebrae 14, 16 as appropriate in light of the condition of vertebrae 14, 16 being treated with injectable material 80. Further, while not intending to be limited to any particular form, it is contemplated that the injection device could be a syringe or pump, although other possibilities also exist.

In one form, injectable material 80 is of a form that can be placed into cancellous or cortical bone. Injectable material 80 may be allowed to solidify to provide structural support and reinforcement. Examples of suitable biocompatible materials for injectable material 80 may include bone cements such as those made from polymethylmethacrylate (PMMA), calcium phosphate, hyrdroxyapatite-tricalcium phosphate (HA-TCP) compounds, bioactive glasses, polymerizable matrix comprising a bisphenol-A dimethacrylate, or CORTOSS® by Orthovita of Malvern, Pa. (generically referred to as a thermoset cortical bone void filler). Calcium sulfate bone void fillers and other filling materials or combinations of filling materials may also be used. Bone void fillers or bone cements may be treated with biological additives such as demineralized bone matrix, collagen, gelatin, polysaccharide, hyaluronic acid, keratin, albumin, fibrin, cells and/or growth factors. Additionally or alternatively, bone void fillers or bone cements may be mixed with inorganic particles such as hydroxyapatite, fluorapatite, oxyapatite, wollastonite, anorthite, calcium fluoride, agrellite, devitrite, canasite, phlogopite, monetite, brushite, octocalcium phosphate, whitlockite, tetracalcium phosphate, cordierite, berlinite or mixtures thereof.

Other osteoinductive, osteoconductive, or carrier materials that may be injected or inserted into vertebral bone include collagen, fibrin, albumin, karatin, silk, elastin, demineralized bone matrix, or particulate bone. Various bone growth promoting biologic materials may also be used including mysenchymal stem cells, hormones, growth factors such as transforming growth factor beta (TGFb) proteins, bone morphogenic proteins (including BMP and BMP2), or platelet derived growth factors. Examples of such materials that can be injected into vertebral bodies are disclosed in U.S. Pub. No. 2005/0267577, the contents of which are hereby incorporated by reference in their entirety.

In one form, injectable material 80 may be used alone such as in vertebroplasty procedures that inject bone cement directly into the interstitial spaces in cancellous bone. Alternatively, the above mentioned bone fillers and treatments may be used with void creation devices such as balloon expansion systems offered by or developed by Kyphon, Inc. of Glendale, Calif. Examples of such systems are disclosed in U.S. Pub. Nos. 2004/0102774 and 20040133280 and U.S. Pat. Nos. 4,969,888 and 5,108,404, all of which are incorporated by reference herein. Other void creation systems that utilize expandable cages or displacement systems may also be used for vertebral body repair. Such systems may be disclosed in U.S. Published Pat. App. No. 2004/0153064 and 2005/0182417 and are incorporated by reference herein.

Following injection of injectable material 80 into vertebrae 14, 16, support body 30 is positioned between vertebrae 14, 16 and engaged with end members 26, 28. More particularly, as illustrated in FIG. 8 the coupling portions of end members 26, 28 are positioned in internal receptacles 63, 65 of coupling members 62, 64 of support body 30. Further, injectable substance 86 is injected into hollow interior 68 of balloon-like structure 56 through an injection device 88 engaged with injection port 66. While not intending to be limited to any particular form, it is contemplated that injection device 88 could be a syringe or pump, although other possibilities for injection device 88 also exist. In one form, support body 30 is coupled with end members 26, 28 before injection of injectable substance 86 is initiated, although forms where support body 30 is at least partially filled with injectable substance 86 before engagement with end members 26, 28 is also contemplated. As support body 30 is filled with injectable substance 86, it expands to provide proper spacing between vertebrae 14, 16 that in one form can correspond to the spacing between vertebrae 14, 16 before removal of vertebra 12. While not shown in FIG. 8, it should be appreciated that a distraction device could also be engaged with vertebrae 14, 16 to provide a desired spacing therebetween as support body 30 is filled with injectable substance 86.

In the illustrated form, ends 58, 60 of balloon-like structure 56 are positioned adjacent to but not in contact with vertebrae 14, 16 once balloon-like structure 56 is expanded. However, in other non-illustrated forms, support body 30 may be provided with a configuration such that ends 58, 60 of balloon-like structure 56 abut against the endplates of vertebrae 14, 16 when support body 30 is in its expanded or inflated configuration. Once support body 30 has been inflated to a desired configuration, injection device 88 can be disengaged from injection port 66 and removed from the surgical site and, if necessary, injection port 66 can be sealed to prevent leakage of injectable substance 86 from support body 30. While not illustrated in FIG. 8, it should also be appreciated that one or more plates, rods, staples or tethers, just to provide a few possibilities, can be engaged with vertebrae 14, 16 to provide additional support thereto and/or assist in maintaining the positioning of implant assembly 30 between vertebrae 14, 16.

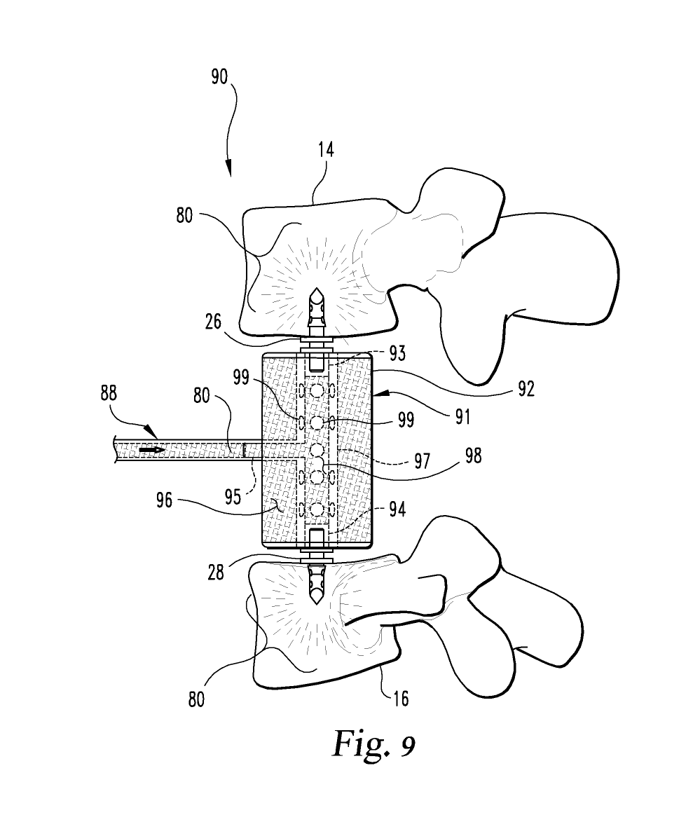

Other configurations for the implant assembly between vertebrae 14, 16 are also possible. For example, turning now to FIG. 9 where like numerals refer to like features previously described, an alternative embodiment implant assembly 90 is illustrated in side plan view. Implant assembly 90 includes first and second end members 26, 28 and a support body 91 formed of an expandable balloon-like structure 92, such as a flexible container or bag, which can be configured the same as balloon-like structure 56 described above unless otherwise noted. Support body 91 further includes a coupling member 93 configured to engage with first end member 26 and a coupling member 94 configured to engage with second end member 28. In addition, balloon-like structure 92 includes a hollow interior 96 within which a support member 97 is positioned. Support member 97 may generally be more rigid than the remainder of balloon-like structure 92, although forms where the rigidity of support member 97 and the remainder of balloon-like structure 92 is the same are also contemplated. A chamber 98 is formed by support member 97 and is in fluid communication with an injection port 95 and coupling members 93, 94 which are further in fluid communication with the hollow interiors of end members 26, 28. Similarly, coupling members 93, 94 can be engaged with end members 26, 28 through a sealed arrangement provided by an o-ring or other sealing mechanism, just to provide a few possibilities. A plurality of apertures 99, only a few of which have been pointed out to preserve clarity, extend through support member 97 such that chamber 98 is also in fluid communication with hollow interior 96 extending around support member 97.

Following engagement of end members 26, 28 with vertebrae 14, 16, support body 91 is engaged with end members 26, 28. Injection device 88 is then coupled with injection port 95 and injectable material 80 is injected through injection port 95 into chamber 98. After injectable material 80 is positioned in chamber 98, it begins to flow through apertures 99 into hollow interior 96 and through coupling members 93, 94 into end members 26, 28 where injectable material 80 is injected into vertebrae 14, 16 through the fenestrations of end members 26, 28. Similarly, injection of injectable material 80 into vertebrae 14, 16 at least partially occurs at the same time support body 91 is inflated with injectable material 80. Once support body 91 has been inflated to a desired configuration, injection device 88 can be disengaged from injection port 95 and removed from the surgical site and, if necessary, injection port 95 can be sealed to prevent leakage of injectable material 80 from support body 91.

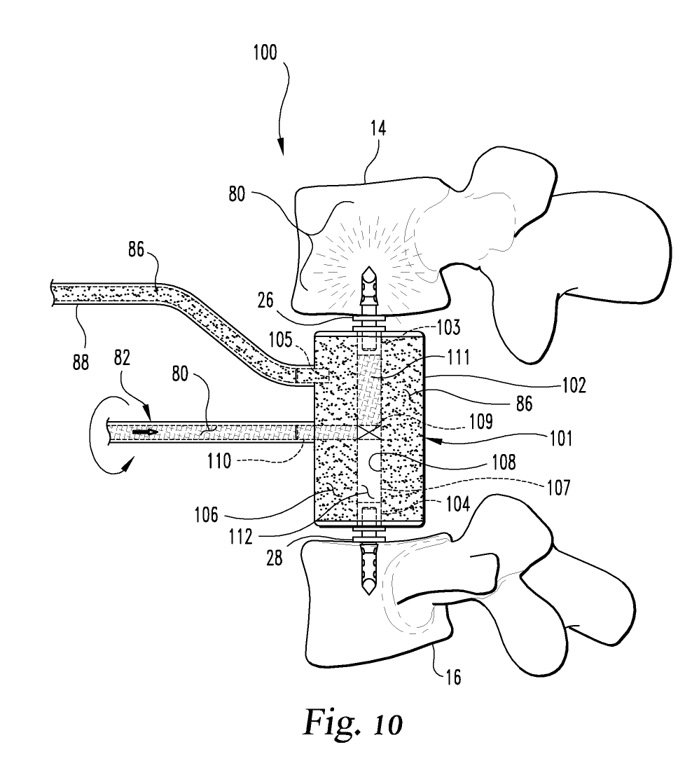

Another alternative embodiment implant assembly 100 is illustrated in a side plan view in FIG. 10, where like numerals refer to like features previously described. Implant assembly 100 includes first and second end members 26, 28 and a support body 101 formed of an expandable balloon-like structure 102, such as a flexible container or bag, which can be configured the same as balloon-like structure 56 described above unless otherwise noted. Support body 101 further includes a coupling member 103 configured to engage with first end member 26 and a coupling member 104 configured to engage with second end member 28. In addition, balloon-like structure 102 includes a hollow interior 106 within which a support member 107 is positioned. Support member 107 may generally be more rigid than the remainder of balloon-like structure 102, although forms where the rigidity of support member 107 and the remainder of balloon-like structure 102 is the same are also contemplated.

A chamber 108 is formed by support member 107 and includes a valve member 109 that separates chamber 108 into an upper portion 111 and a lower portion 112. Upper and lower portions 111, 112 are in fluid communication with coupling members 103, 104, respectively, which are further in fluid communication with end members 26, 28, respectively. Similarly, coupling members 103, 104 can be engaged with end members 26, 28 through a sealed arrangement provided by an o-ring or other sealing mechanism, just to provide a few possibilities. A pathway 110 extends through hollow interior 106 into communication with valve member 109 in order to facilitate control of valve member 109 and selective injection of injectable material 80 into upper and lower portions 111, 112. In one form, valve member 109 is configured to facilitate injection of injectable material 80 into upper and lower portions 111, 112 at the same time and to facilitate selective injection of injectable material 80 into only one of upper and lower portions 111, 112 at a time. Once injectable material 80 is allowed to enter one or both of upper and lower portions 111, 112, it can be passed through one or both of end members 26, 28 into one or both of vertebrae 14, 16. For example, as illustrated in FIG. 10 an injection device 82 is engaged with valve member 109 through pathway 110 and injectable material 80 is being directed to upper portion 111, through coupling member 103 and into vertebra 14 through end member 26. It is contemplated that injection device 82 could be a syringe or pump, although other possibilities for injection device 82 also exist. Once a desired amount of injectable material 80 has been injected into vertebra 14, valve member 109 can be adjusted to prevent further passage of injectable material 80 into upper portion 111 and/or to facilitate passage of injectable material 80 into lower portion 112 for injection into vertebrae 16 through end member 28 if desired. Once the desired amount of injectable material 80 has been delivered to one or both of vertebrae 14, 16, injection device 82 can be disengaged from support body 101 and removed from the surgical site.

Support body 101 also includes an injection port 105 in fluid communication with hollow interior 106 and through which injectable substance 86 can be injected into hollow interior 106 from injection device 88. As injectable substance 86 is injected into hollow interior 106, it fills the space surrounding support member 107 and can inflate or expand support body 101 to provide a desired spacing between vertebrae 14, 16. While not previously discussed, it should be appreciated that injectable substance 86 can be injected into support body 101 simultaneous with, before, or after injection of injectable material 80. In addition, it should be appreciated that injectable material 80 and injectable substance 86 can be the same, or they can be different from one another. In one form where injectable material 80 and injectable substance 86 are different from one another they can be selectively chosen to impart different characteristics, such as rigidity, on the respective portions of support body 101 in which they are contained. Once support body 101 has been inflated to a desired configuration, injection device 88 can be disengaged from injection port 105 and removed from the surgical site and, if necessary, injection port 105 can be sealed to prevent leakage of injectable material 80 from support body 101.

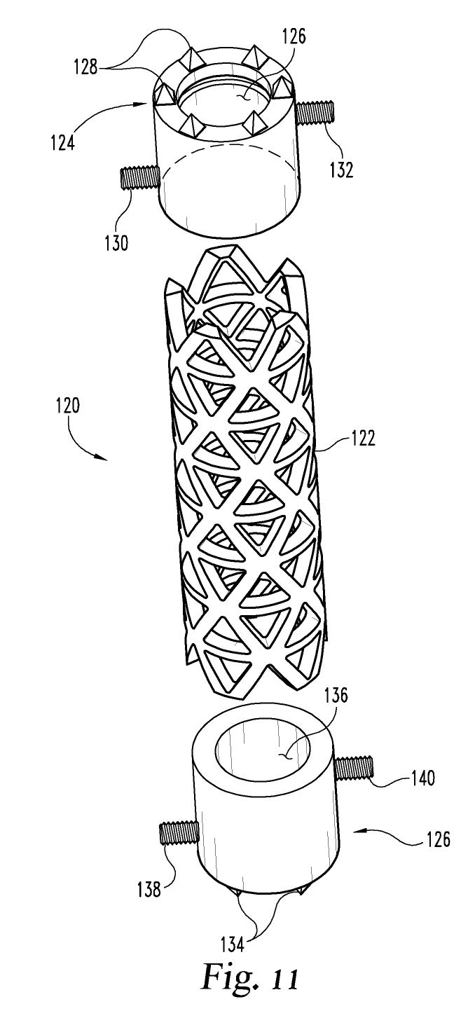

Use of end members 26, 28 with support bodies that do not include a balloon-look structure are also possible, one non-limiting example of which is illustrated in perspective view in FIG. 11. More particularly, support body 120 includes a mesh cage structure 122 which can be formed of a rigid or elastic material. Support body 120 also includes a first end cap 124 and a second end cap 126. End cap 124 includes a plurality of bone engaging features 128 in the form of spikes or teeth configured to engage with vertebra 14. End cap 124 also includes an upper receptacle 126 configured to receive coupling portion 44 of end member 26 and a lower receptacle (not shown) configured to receive a portion of mesh cage structure 122. A transverse set screw 130 extends through end cap 124 into communication with the lower receptacle and is configured to bear against the portion of mesh cage structure 122 positioned therein in order to lock end cap 124 and mesh cage structure 122 in engagement with one another. Similarly, a transverse set screw 132 extends through end cap 124 into communication with upper receptacle 126 and is configured to bear against coupling portion 44 of end member 26 positioned therein in order to lock end cap 124 and end member 26 in engagement with one another.

Similar to end cap 124, end cap 126 also includes a plurality of bone engaging features 134 in the form of spikes or teeth configured to engage with vertebra 16. End cap 126 also includes an upper receptacle 136 configured to receive a portion of mesh cage structure 122 and a lower receptacle (not shown) configured to receive the coupling portion of end member 28. A transverse set screw 140 extends through end cap 126 into communication with upper receptacle 136 and is configured to bear against the portion of mesh cage structure 122 positioned therein in order to lock end cap 126 and mesh cage structure 122 in engagement with one another. Similarly, a transverse set screw 138 extends through end cap 126 into communication with the lower receptacle and is configured to bear against the coupling portion of end member 28 positioned therein in order to lock end cap 126 and end member 28 in engagement with one another.

When support body 120 is used in connection with end members 26, 28, end members 26, 28 may be first engaged with vertebrae 14, 16 and injectable material 80 may then be injected through end members 26, 28 into vertebrae 14, 16 as discussed above. Following injection of injectable material 80, support body 120 can be positioned between vertebrae 14, 16 and end caps can be engaged with end members 26, 28. Transverse set screws 130, 132, 138, 140 can then be adjusted to lock the assembly of end members 26, 28, end caps 124, 126, and mesh cage structure 122 together. In addition to support body 120, other alternative embodiment support bodies are also usable in connection with end members 26, 28, non-limiting examples of which are illustrated and described in U.S. Pat. Nos. 5,702,453, 5,776,197, 5,776,198, 6,344,057, 7,238,205, and 7,621,953, and in U.S. Patent Publication Nos. 2010/0249934, 2010/0114319, 2007/0255408, 2008/0177387, the contents of which are incorporated herein by reference in their entirety.

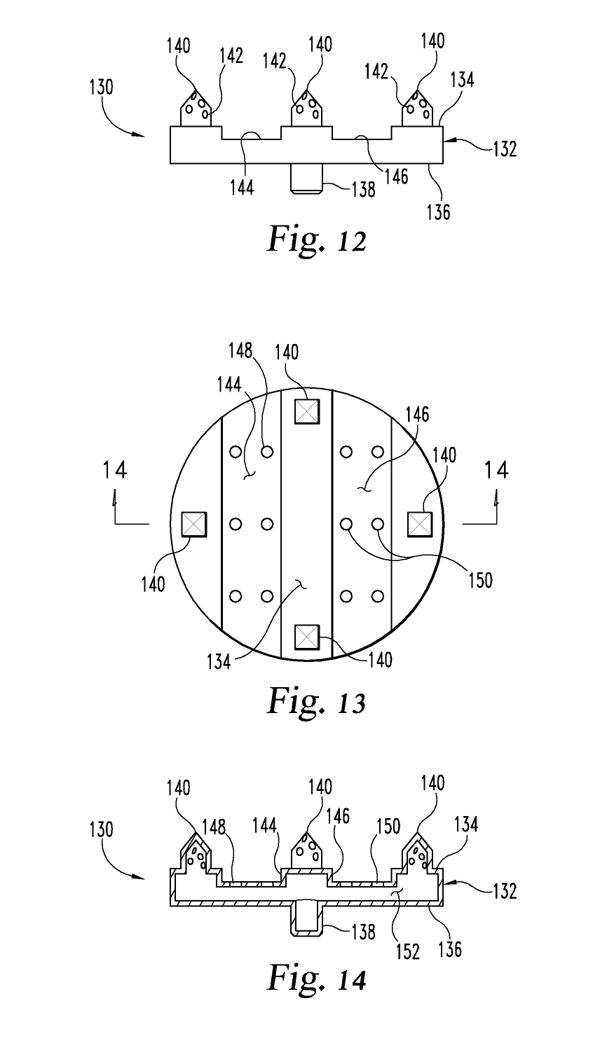

While alternative arrangements for support body 30 have been discussed above, it should also be appreciated that alternative configurations for end members 26, 28 are also possible. For example, one non-limiting alternative embodiment end member 130 will be described in connection with FIGS. 12-14. End member 130 includes a base portion or plate member 132 which can generally be configured to extend partially or entirely across the endplate of an adjacent vertebra. Plate member 132 extends between a first surface 134 and a second surface 136 from which a coupling portion 138 extends. Coupling portion 138 is generally configured to engage with a coupling member of a support body. Plate member 132 also includes a plurality of bone engaging members 140 in the form of teeth or spikes, although other configurations for bone engaging members 140 are also contemplated, that extend from first surface 134. Each of bone engaging members 140 includes a plurality of fenestrations 142 (only a few of which have been indicated to preserve clarity) that communicate with a hollow interior 152 of plate member 132. Hollow interior 152 opens through coupling portion 138 such that an injection port through which a material can be injected into end member 130 is provided, although arrangements where the injection port is positioned elsewhere are also possible.

Plate member 132 also includes bone graft receptacles 144, 146 which are formed in first surface 134 such that they are offset toward second surface 136. Bone graft receptacles 144, 146 are generally configured to receive a bone graft or other bone-growth promoting material and position such bone graft or other bone-growth promoting material against or in close proximity to the endplate of an adjacent vertebra. In other non-illustrated forms, plate member 132 may also be provided without bone graft receptacles 144, 146. Bone graft receptacles 144, 146 each include a plurality of fenestrations 148, 150 (only a few of which have been indicated to preserve clarity), respectively, that communicate with hollow interior 152 to facilitate bony ingrowth into plate member 132 or through plate member 132 and across space 22 between vertebrae 14, 16 to provide a bony fusion therebetween. In other non-illustrated forms, fenestrations 148, 150 are absent from bone graft receptacles 144, 146.

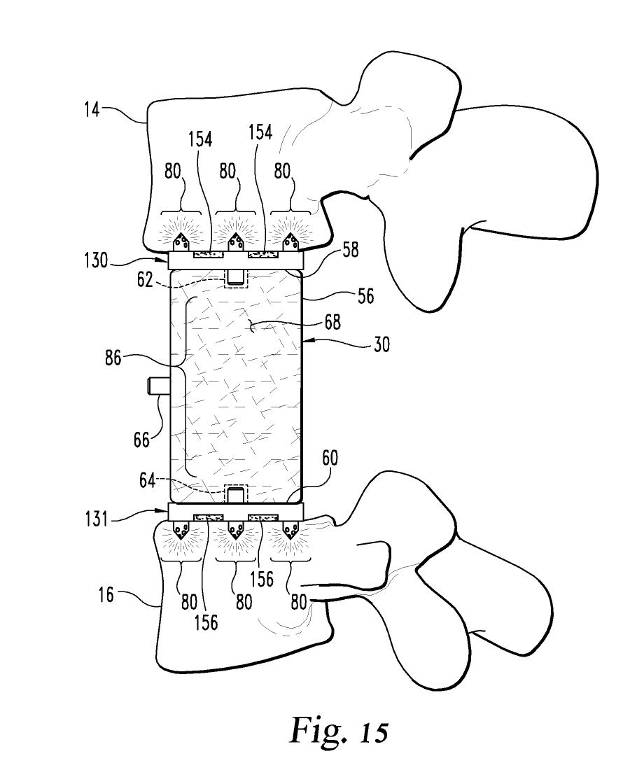

Referring now to FIG. 15, where like numerals refer to like features previously described, end member 130 and an end member 131 configured the same as end member 130 described above are engaged with vertebrae 14, 16. In other forms, use of only one of end members 130, 131 in combination with an alternatively arranged end member is also possible. End members 130, 131 can be positioned between and engaged with vertebrae 14, 16 in a manner similar to that described above in regard to the placement of end members 26, 28. Moreover, in the illustrated form bone graft or bone-growth promoting material 154 is positioned between end member 130 and vertebra 14 and in contact with the endplate of vertebra 14. Similarly, bone graft or bone-growth promoting material 156 is positioned between end member 131 and vertebra 16 and in contact with the endplate of vertebra 16. Following engagement of end members 130, 131 with vertebrae 14, 16, insertion device 70 (if used) or another injection device (not shown) can be used to inject injectable material 80 into the hollow interiors of end members 130, 131 and through the fenestrations of the bone engaging members into vertebrae 14, 16. While not previously discussed, it should be appreciated that end members 130, 131 may include a self-sealing valve (not specifically shown) that prevents injectable material 80 from leaking therefrom.

Once a desired amount of injectable material 80 has been injected into vertebrae 14, 16, support body 30 can be positioned between vertebrae 14, 16 and engaged with end members 130, 131 by positioning the coupling portions of end members 130, 131 in the coupling members 62, 64 as shown in FIG. 15. Support body 30 can then be inflated or expanded by injection of injectable substance 86 into hollow interior 68 of balloon-like structure 56 through injection port 66. In one form, support body 30 is coupled with end members 130, 131 before injection of injectable substance 86 is initiated, although forms where support body 30 is at least partially filled with injectable substance 86 before engagement with end members 130, 131 is also contemplated. As support body 30 is filled with injectable substance 86, it expands to provide proper spacing between vertebrae 14, 16 that in one form can correspond to the spacing between vertebrae 14, 16 before removal of vertebra 12. While not shown in FIG. 15, it should be appreciated that a distraction device could also be engaged with vertebrae 14, 16 to provide a desired spacing therebetween as support body 30 is filled with injectable substance 86. In addition, as shown in FIG. 15, ends 58, 60 of balloon-like structure 56 bear against the surfaces of end members 130, 131 facing support body 30. It should also be appreciated that in addition to use with support body 30, end members 130, 131 may also be used in connection with the alternative embodiment support bodies described herein above.

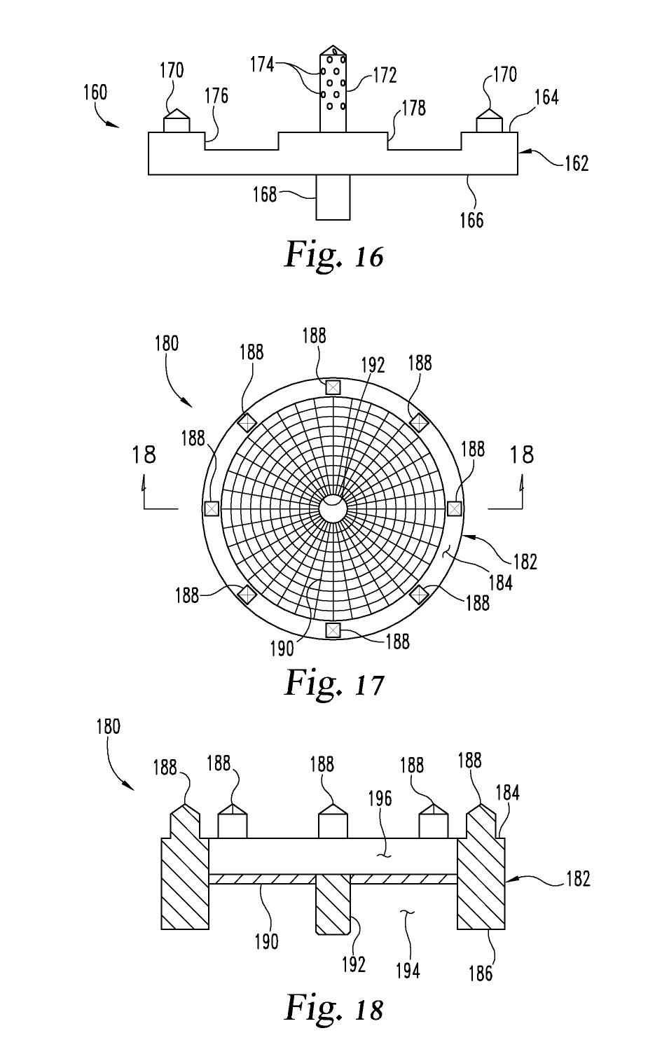

Another alternative embodiment end member 160 configured to be used with one or more of the support bodies described herein is illustrated in FIG. 16. End member 160 includes a base portion or plate member 162 which can generally be configured to extend partially or entirely across the endplate of an adjacent vertebra. Plate member 162 extends between a first surface 164 and a second surface 166 from which a coupling portion 168 extends. Coupling portion 168 is generally configured to engage with a coupling member of a support body. Plate member 162 also includes a plurality of bone engaging members 170, 172 in the form of teeth or spikes, although other configurations for bone engaging members 170, 172 are also contemplated, that extend from first surface 164. As illustrated in FIG. 16, bone engaging member 174 is substantially longer than bone engaging members 170 and, in one form, can be configured to extend into an adjacent vertebrae to at least a midpoint between the endplates of the vertebra. Bone engaging member 172 also includes a plurality of fenestrations 174 (only a few of which have been indicated to preserve clarity) that communicate with a hollow interior (not shown) of plate member 162. The hollow interior of plate member 162 opens through the end of coupling portion 168 such that an injection port through which a material can be injected into end member 160 is provided, although arrangements where the injection port is positioned elsewhere are also possible. Plate member 162 also includes bone graft receptacles 176, 178 which are formed in first surface 164 such that they are offset toward second surface 166. Bone graft receptacles 176, 178 are generally configured to receive a bone graft or other bone-growth promoting material and position such bone graft or other bone-growth promoting material against or in close proximity to the endplate of an adjacent vertebra. In other non-illustrated forms, plate member 162 may also be provided without bone graft receptacles 176, 178, and (when present) bone graft receptacles 176, 178 may also include fenestrations that communicate with the hollow interior of plate member 162.

End member 160 can be positioned between a pair of adjacent vertebrae and engaged with one of the vertebrae in a manner similar to that described above in regard to the placement of end members 26, 28. Moreover, in the illustrated form bone graft or bone-growth promoting material can be positioned between end member 160 and the adjacent vertebra and in contact with the endplate of the vertebra. Following engagement of end member 160 with the adjacent vertebra, insertion device 70 (if used) or an injection device (not shown) can be used to inject injectable material 80 into the hollow interior of end member 162 and through fenestrations 174 of bone engaging member 172 into the adjacent vertebra. While not previously discussed, it should be appreciated that end member 160 may include a self-sealing valve (not specifically shown) that prevents injectable material 80 from leaking therefrom. Following injection of a desired amount of material 80, a support body may be positioned between the vertebrae and engaged with end member 160, although forms where material 80 is injected following engagement of a support body with end member 160 are also possible.

Another alternative embodiment end member 180 configured to be used with one or more of the support bodies described herein is illustrated in FIGS. 17-18. End member 180 includes a sidewall member 182 which can generally be configured to extend partially or entirely across the endplate of an adjacent vertebra. Sidewall member 182 extends between a first surface 184 and a second surface 186 and defines a generally hollow interior 194 that extends between and opens at first surface 184 and second surface 186. End member 180 also includes a plurality of bone engaging members 188 in the form of teeth or spikes, although other configurations for bone engaging members 188 are also contemplated, that extend from first surface 184. A mesh material 190 extends across hollow interior 194 and includes a center hub 192 which is generally configured to engage with a coupling member of a support body. As illustrated in FIG. 18, mesh material 190 is positioned between first surface 184 and second surface 186 such that a cavity or receptacle 196 is formed below first surface 184. Receptacle 196 is generally configured to receive a bone graft or other bone-growth promoting material and position such bone graft or other bone-growth promoting material against or in close proximity to the endplate of an adjacent vertebra. Due to the open nature of mesh material 190, bone or bony tissue can grow therethrough to incorporate end member 180 with the adjacent vertebra and/or to attach to the support body with which end member 180 is engaged in order to facilitate bony fusion between the adjacent vertebrae.

End member 180 can be positioned between a pair of adjacent vertebrae and engaged with one of the vertebrae in a manner similar to that described above in regard to the placement of end members 26, 28. Moreover, in the illustrated form bone graft or bone-growth promoting material can be positioned between end member 180 and the adjacent vertebra and in contact with the endplate of the vertebra. Following engagement of end member 180 with the adjacent vertebra, a support body can be positioned between the vertebrae and engaged with end member 180 as discussed above.

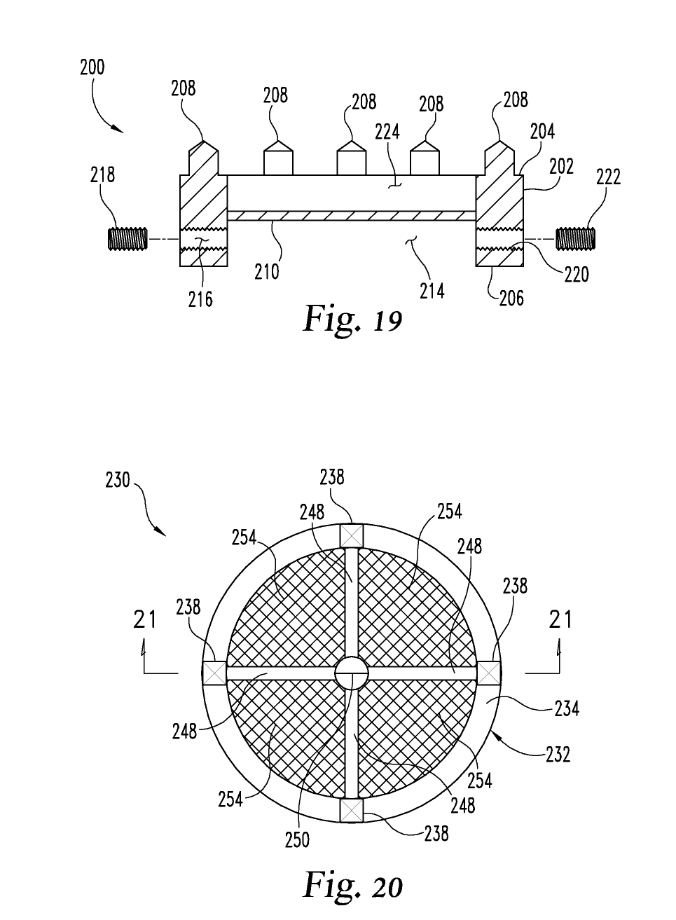

With particular reference now to FIG. 19, another alternative embodiment end member 200 configured to be used with one or more of the support bodies described herein is illustrated. End member 200 includes a sidewall member 202 which can generally be configured to extend partially or entirely across the endplate of an adjacent vertebra. Sidewall member 202 extends between a first surface 204 and a second surface 206 and defines a generally hollow interior 214 that extends between and opens at first surface 204 and second surface 206. End member 200 also includes a plurality of bone engaging members 208 in the form of teeth or spikes, although other configurations for bone engaging members 208 are also contemplated, that extend from first surface 204. A mesh material 210 extends across hollow interior 214 and is positioned between first surface 204 and second surface 206 such that a cavity or receptacle 224 is formed below first surface 204. Receptacle 224 is generally configured to receive a bone graft or other bone-growth promoting material and position such bone graft or other bone-growth promoting material against or in close proximity to the endplate of an adjacent vertebra. Due to the open nature of mesh material 210, bone or bony tissue can grow therethrough to incorporate end member 200 with the adjacent vertebrae and/or to attach to the support body with which end member 200 is engaged in order to facilitate bony fusion between the adjacent vertebrae.

End member 200 is further configured to receive a portion of a support body in hollow interior 214 below mesh material 210. Transverse bores 216, 220 extend through sidewall member 202 and are configured to receive transverse set screws 218, 222 such that set screws 218, 222 can bear against the portion of the support body received in hollow interior 214 in order to lock end member 200 with the support body. End member 200 can be positioned between a pair of adjacent vertebrae and engaged with one of the vertebrae in a manner similar to that described above in regard to the placement of end members 26, 28. Moreover, in the illustrated form bone graft or bone-growth material can be positioned between end member 200 and the adjacent vertebra and in contact with the endplate of the vertebra. Following engagement of end member 200 with the adjacent vertebra, a support body can be positioned between the vertebrae and engaged with end member 200 as discussed above.

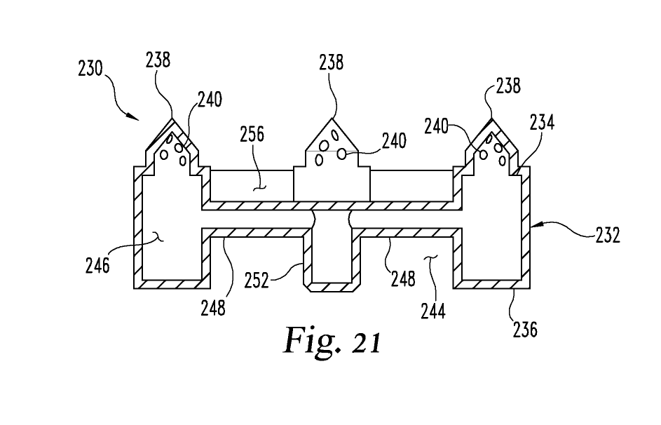

Another alternative embodiment end member 230 configured to be used with one or more the support bodies described herein is illustrated in FIGS. 20 and 21. End member 230 includes a sidewall member 232 which can generally be configured to extend partially or entirely across the endplate of an adjacent vertebra. Sidewall member 232 extends between a first surface 234 and a second surface 236 and defines a generally hollow interior 244 that extends between and opens at first surface 234 and second surface 236. End member 230 also includes a plurality of bone engaging members 238 in the form of teeth or spikes, although other configurations for bone engaging members 238 are also contemplated, that extend from first surface 234. Each of bone engaging members 238 includes a plurality of fenestrations 240 (only a few of which have been indicated to preserve clarity) that communicate with a hollow interior 246 of end member 230. Hollow interior 246 opens through a coupling portion 252 such that an injection port through which a material can be injected into end member 230 is provided, although arrangements where the injection is positioned elsewhere are also possible.

End member 230 also includes a plurality of hollow struts 248 which extend from sidewall member 232 across hollow interior 244 to a center hub 250. As illustrated in FIG. 21, coupling portion 252 which is generally configured to engage with a coupling member of a support body extends from center hub 250. In addition, as also illustrated in FIG. 21 struts 248 are positioned below first surface 234 of sidewall member 232. A mesh material 254 extends across hollow interior 244 between struts 248 and is positioned between first surface 234 and second surface 236 such that a cavity or receptacle 256 is formed below first surface 234. Receptacle 256 is generally configured to receive a bone graft or other bone-growth promoting material and position such bone graft or other bone-growth promoting material against or in close proximity to the endplate of an adjacent vertebra. Due to the open nature of mesh material 254, bone or bony tissue can grow therethrough to incorporate end member 230 with the adjacent vertebrae and/or to attach to the support body with which end member 230 is engaged in order to facilitate bony fusion between the adjacent vertebrae.

End member 230 can be positioned between a pair of adjacent vertebrae and engaged with one of the vertebrae in a manner similar to that described above in regard to the placement of end members 26, 28. Moreover, in the illustrated form bone graft or bone-growth promoting material can be positioned between end member 230 and the adjacent vertebra and in contact with the endplate of the vertebra. Following engagement of end member 230 with the adjacent vertebra, insertion device 70 (if used) or an injection device (not shown) can be used to inject injectable material 80 into hollow interior 246 and through struts 248 of end member 232 until material 80 exits fenestrations 240 of bone engaging members 238 into the adjacent vertebra. While not previously discussed, it should be appreciated that end member 232 may include a self-sealing valve (not specifically shown) that prevents injectable material 80 from leaking therefrom. Following engagement of end member 230 with the adjacent vertebra and injection of injectable material 80 in the adjacent vertebra, a support body may be positioned between the vertebrae and engaged with end member 230 as discussed above.

While not previously discussed, it should be appreciated that, unless otherwise described, the components of the implant assemblies described herein may be made from any suitable biocompatible material, including but not limited to titanium, titanium alloy, stainless steel, metallic alloys, polyaryletherketone (PAEK), polyetheretherketone (PEEK), carbon-reinforced PEEK, polyetherketoneketone (PEKK), polysulfone, polyetherimide, polyimide, ultra-high molecular weight polyethylene (UHMWPE), and plastics, just to name a few possibilities. Further, it should also be appreciated that the instruments, devices, systems, techniques and methods described herein may also be used in surgical procedures involving animals, or in demonstrations for training, education, marketing, sales and/or advertising purposes. Furthermore, the instruments, devices, systems, techniques and methods described herein may also be used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present application and is not intended to make the present application in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the application, that scope being defined by the claims that follow. In reading the claims it is intended that when words/phrases such as “a”, “an”, “at least one”, and/or “at least a portion” are used, there is no intention to limit the claim to only one item unless specifically stated to the contrary in the claim. Further, when the language “at least a portion” and/or “a portion” is used, the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the application as defined herein or by any of the following claims are desired to be protected.

What is claimed is:

1. An implant assembly configured to be positioned between a first vertebra and a second vertebra, comprising:
a first end member including at least one bone engaging portion configured to engage with the first vertebra;
a second end member including at least one bone engaging portion configured to engage with the second vertebra; and
a support body configured to axially extend between and engage with said first and second end members;
wherein at least said first end member includes a first injection port in fluid communication with said at least one bone engaging portion, said at least one bone engaging portion of said first end member including a number of fenestrations through which a material injected into said first injection port is deliverable into the first vertebra.

2. The implant assembly of claim 1, wherein said second end member further includes a second injection port in fluid communication with said at least one bone engaging portion, said at least one bone engaging portion of said second end member including a number of fenestrations through which a material injected into said second injection port is deliverable into the second vertebra.

3. The implant assembly of claim 2, wherein said support body includes an inflatable bag member, said bag member including a first coupling member engageable with said first injection port and a second coupling member engageable with said second injection port.

4. The implant assembly of claim 3, wherein said bag member includes a first chamber and a second chamber extending around said first chamber, said first chamber being configured to direct one or more materials from a delivery device into said first injection port, said second injection port, and said second chamber.

5. The implant assembly of claim 3, wherein said bag member includes a first chamber and a second chamber extending around said first chamber, said first chamber including a valve member operable to selectively direct one or more materials from a delivery device to one or both of said first injection port and said second injection port.

6. The implant assembly of claim 1, wherein said support body extends along a longitudinal axis between a first end and a second end and is axially adjustable to alter a distance between said first end and said second end.

7. The implant assembly of claim 1, wherein said at least one bone engaging portion of said first end member includes a plurality of bone engaging members in fluid communication with said first injection port, each of said plurality of bone engaging members including a number of fenestrations through which said material injected into said first injection port is deliverable into the first vertebra.

8. The implant assembly of claim 1, wherein said first end member includes a first end wall from which said at least one bone engaging portion extends, said first end wall including one or more receptacles formed therein adjacent to said at least one bone engaging portion, said one or more receptacles being configured to receive a bone growth promoting material.

9. The implant assembly of claim 1, wherein said material is selected from the group consisting of polymethylmethacrylate (PMMA), calcium phosphate, hydroxyapatite-tricalcium phosphate (HA-TCP) compounds and bioactive glasses.

10. The implant assembly of claim 1, wherein said first end member includes a base portion from which said at least one bone engaging portion extends, said base portion including a hollow interior in fluid communication with said first injection port and a number of fenestrations in communication with said hollow interior through which said material injected into said first injection port is deliverable to a location adjacent an endplate of the first vertebra.

11. The implant assembly of claim 1, wherein said first end member includes a base portion from which said at least one bone engaging portion extends, said base portion being configured to be positioned against a first endplate of the first vertebra and said at least one bone engaging portion being configured to extend to a location adjacent to a point positioned midway between a first endplate and a second endplate of the first vertebra when said base portion is positioned against the first endplate of the first vertebra.

12. The implant assembly of claim 1, wherein each of said first and second end members includes a base portion and said at least one bone engaging portion of at least one of said first and second end members includes a plurality of bone engaging members extending from said base portion.

13. An implant assembly configured to be positioned between a first vertebra and a second vertebra, comprising:
a first end member including at least one bone engaging member configured to engage with the first vertebra;
a second end member including at least one bone engaging member configured to engage with the second vertebra; and
a support body configured to axially extend between and engage with said first and second end members;
wherein at least said first end member includes a base portion extending between a first side facing said second end member and a second side from which said at least one bone engaging member extends, said base portion including a hollow interior extending between and opening at said first and second sides and a mesh material extending across said hollow interior.

14. The implant assembly of claim 13, wherein said second end member includes a base portion extending between a third side facing said first end member and a fourth side from which said at least one bone engaging member extends, said base portion including a hollow interior extending between and opening at said third and fourth sides and a mesh material extending across said hollow interior.

15. The implant assembly of claim 13, wherein said mesh material is positioned between said first and second sides of said base portion of said first end member.

16. The implant assembly of claim 13, wherein said mesh material includes a first coupling portion configured to be engaged with said support body.

17. The implant assembly of claim 16, wherein said support body is an inflatable bag member including a second coupling portion configured to engage with said first coupling portion of said first end member.

18. The implant assembly of claim 13, wherein said base portion includes a sidewall extending around said hollow interior, and a plurality of struts extend from said sidewall and through said hollow interior to a hub member.

19. The implant assembly of claim 18, wherein said hub member includes an injection port in fluid communication with said at least one bone engaging member, said at least one bone engaging member of said first end member including a number of fenestrations through which a material injected into said first injection port is deliverable into the first vertebra.

20. The implant assembly of claim 18, wherein said hub member is configured to engage with a first end of said support body.

21. The implant assembly of claim 13, wherein said base portion includes a sidewall extending around said hollow interior, said sidewall being configured to engage with a first end of said support body.

22. A method for positioning an implant between a first vertebra and a second vertebra, comprising:
providing a first end member including at least one bone engaging portion configured to engage with the first vertebra, a second end member including at least one bone engaging portion configured to engage with the second vertebra, and a support body configured to axially extend between and engage with said first and second end members;
positioning said first end member into a space between the first vertebra and the second vertebra and engaging the first end member with the first vertebra;
positioning said second end member into the space between the first vertebra and the second vertebra and engaging said second end member with the second vertebra;
injecting a material into said first end member and delivering said material into the first vertebra through said at least one bone engaging portion of said first end member; and
positioning said support body between and into engagement with said first and second end members.

23. The method of claim 22, which further includes injecting said material into said second end member and delivering said material into the second vertebra through said at least one bone engaging portion of said second end member.

24. The method of claim 22, which further includes positioning a bone growth promoting material between said first end member and the first vertebra and said second end member and the second vertebra.

25. The method of claim 22, which further includes injecting an injectable substance into said support body.

26. The method of claim 25, wherein injecting said material into said first end member occurs upon injecting said injectable substance into said support body.

27. The method of claim 22, which further includes adjusting a length of said support body extending between said first and second end members following engagement of said support body with said first and second end members.
